(19) United States
(12) Patent Application Publication
(10) Pub. No.: US 2024/0016725 A1
(43) Pub. Date: Jan. 18, 2024

(54) SOLIDIFIED LIP GLOSS STICK-TYPE COSMETIC COMPOSITION STABILIZING HIGH-CONTENT OLIGOMER-TYPE PHYTOSTEROL ESTERS

(71) Applicant: C&C INTERNATIONAL CO., LTD., Gyeonggi-do (KR)

(72) Inventors: Jong Taeg YANG, Cheongju-si Chungcheongbuk-do (KR); Seok Hwan KWON, Hwaseong-si, Gyeonggi-do (KR); Eun Chul BAE, Suwon-si,Gyeonggi-do (KR)

(21) Appl. No.: 18/038,501

(22) PCT Filed: Oct. 29, 2021
(86) PCT No.: PCT/KR2021/015448

§ 371 Date: May 24, 2023

(30) Foreign Application Priority Data
Jun. 28, 2021 (KR) 10-2021-0083693

(51) Int. Cl. A61K 8/92; A61K 8/02; A61Q 1/06

(52) U.S. Cl. CPC A61K 8/92; A61K 8/0229; A61Q 1/06; A61K 2800/80; A61K 2800/43; A61K 2800/522

(57) ABSTRACT

The present invention is related to novel lip gloss-type cosmetic composition comprising (a) hydrocarbon-based wax purified from natural gas or petroleum with a melting point of 85° C. or higher, (b) oligomer-type phytosterol ester, (c) fatty acid complex ester oil, (d) polymeric film forming agent, and (e) a gelling agent including an organic modified clay mineral, which is in a solid stick form having high gloss and a sense of use such as liquid or semi-solid lip gloss.

### TECHNICAL FIELD

The present invention relates to a solidified lip gloss stick-type cosmetic composition stabilizing high-content oligomer-type phytosterol esters.

### BACKGROUND ART

Generally, a lip gloss is a lip cosmetic composition which nourishes the lips or maintains gloss, and is applied directly to the lips or after applying lipstick. Such lip gloss is provided as a form of viscous liquid form or a soft semi-solid form and the lip gloss in a solid stick form prepared from the mixture of various waxes, oils and pigments is more commonly used among them.

The semi-solid or solid stick type lip gloss is prepared by steps of storing the lip gloss composition in which wax and oil are the main components in a container and then naturally cooling or rapidly cooling the lip gloss composition to solidify it.

Although solid stick lip gloss is more convenient and easy to manage than viscous liquid or soft semi-solid for lip makeup, it is difficult to prepare solid stick lip gloss which has high gloss that can be implemented in liquid or semi-solid products while having moderate hardness and of which structure is a form of glossy stick type form with minimal hardness.

A typical lip gloss stick is manufactured by dispersing a pigment (0.1-10%) in an oil-phase of oily base (20-90%) and mixing it with a wax-phase material (5-20%), which shows a difference in the sense of use due to hardness and productivity depending on the mixing ratio of wax and oil.

However, such a lip gloss stick has a problem in that when the hardness is too high, the solidified stick does not be spread well or lacks oil gloss, and if the hardness is too low, the oil gloss is excellent, but their productivity are reduced due to poor moldability with the stick. Moreover, under the storage temperature condition, the solid stick melts or oil is separated, so the hardness in the early stage of use is not maintained over time and the property of the stick is degraded

There has been lots of studies and research have been performed in various fields to solve those problems, and there has been needed to develop a new product such as a lip gloss stick which provides high gloss with an oily texture when applied to a lip and provides convenience and excellent stability when portable and used.

Korea Patent Publication No. 10-2018-0028512 (A) and PCT/WO 2017008243 (A1) discloses a solid anhydrous composition with a combination of waxes, comprising, a) at least one wax with a melting point greater than or equal to 60° C.; b) at least one wax, different from a) , of solid linear ester derived from C₆-C₃₀ fatty acid, c) at least one nonvolatile oil, and d) at least one pasty compound, wherein the weight ratio of a) and b) is from 0.05 to 4.

Korea patent Publication No. 10-2021-0051331 (A) discloses a lip cosmetic composition comprising a silicone gel, silicone oil, silicone powder, wax and pigment, which shows little glossy, good spreading ability, effectively covering activity on wrinkles on the lips after application, and improving a lip dryness.

Korea Patent Publication No. 10-2021-0051024 (A) discloses a high gloss lip make-up composition improving sense of gloss and sticky problem of lip, comprising, (a) at least one silicone resin (phenylpropyl dimethyl siloxy silicate) 1-5%, (b) at least one silicone oil (dimethyl dimethicone) 1-10%, (c) at least one wax (polyethylene, micro-crystalline wax, synthetic wax/ethylene/propylene copolymer) and (d) at least one hydrocarbon oil and ester oil.

Korea Patent Registration No. 10-0876264 (B) and PCT/WO2006017203 (A1) discloses a high gloss and non-feathering lip stick composition comprising (a) at least one fatty alcohol soluble polysaccharide polymer including C1-C8 alkyl-modified polysaccharide polymer and a liquid polyol ester including pentaerythrityl adipate/caprate/caprylate/heptanoate (b) at least one wax including polyethylene and micro-crystalline wax, (c) sugar fatty acid ester including methyl glucose sesquistearates; and at least one hydrocarbon polymer and a pigment.

In order to obtain the stick-type solid composition, the components consisting of at least one natural wax, synthetic wax, and high-grade fatty alcohol are generally mixed together to be structured in a stick form, or a paste-type polymer substance or oil thickening substance is mixed in vegetable oil, hydrocarbon oil, and synthetic ester oil to be structured in a semi-solid form. Those solid-type compositions do not flow, particularly, which is stable at room temperature

In order to differentiate the lip gloss formulation with excellent gloss and soft use from the previous lipstick formulation, when wax component is not mixed or too much oil is mixed in a general stick-type solid composition, the final product does not provide enough hardness to prevent from the flow of lipstick at room temperature and to maintain the stick during all the procedure.

The lipstick shape is convenient to use makeup on the lips, but it has a disadvantage that it is less glossy than the paste lip gloss shape.

The viscosity increases too high at a high temperature where a large amount of the paste-phase polymer substance or the oil-thickening agent substance being used in the known art, is mixed and thus it is difficult to form a stick shape and it is very sticky when used.

In particular, when a high content of the oligomer-type sterol ester is prescribed due to a low melting point of about 38° C., it give rise to several problems such as deteriorated final product in terms of aesthetic appearance due to sweating of the surface.

Additionally, when hydrocarbon-based wax, synthetic wax, or vegetable wax purified from natural gas or petroleum oil, is applied to stabilize high viscosity oils, an additionally prescribed with oligomeric sterol esters, it causes to some problems, for example, a lack of gloss and thickness due to too hard or thin on the lips when it is spread.

The gloss expressed after the application on the lips of the above lip stick in the case of mixing materials such as silicone polymer, Vaseline, shea butter or the like, can be significantly reduced in comparison with the inventive lipstick composition of the present invention.

Although oligomer-type phytosterol esters used as main raw materials in the prior art have been widely used to improve moisturizing effects and the dispersibility of pigment in conventional lipstick compositions, the present inventors have found that they are not satisfactory with the respect to the stability in high temperature and molding when applying high-content oligomer-type phytosterol esters.

However, there have been no suggestion or disclosure on the lip gloss stick-type composition in which high-content oligomer-type phytosterol esters are structurally stabilized with minimal hardness sticks, in any of above cited literatures, the disclosures of which are incorporated herein by reference.

### DISCLOSURE OF INVENTION

### Technical Problem

To solve the previous problems, the present inventors have tried to find a cosmetic composition for lips with excellent applicability due to its glossy and soft texture with solving the previous problems and the inventive lip stick composition showed the superior glossy effect and soft use of solid sticks compared to conventional solid lipstick contents as well as the superior convenient effect on using stick products compared to liquid or semi-solid lip gloss contents when applied to lips, which is confirmed by several tests for example, on the sensual evaluation test of gloss and long-term stability by temperature, such as (1) Comparative measurement of sensual evaluation items (Experimental Example 1), (2) Comparative test on stability test (Experimental Example 2), and (3) Determination on compatibility of each ingredient (Experimental Example 3) finally.

### Solution to Problem

The technical solution to solve the problem of the background art is for the development of novel solidified lip gloss stick-type cosmetic composition stabilizing high-content oligomer-type phytosterol esters.

Accordingly, it is an object of the present invention to provide a solidified lip gloss stick-type cosmetic composition stabilizing high-content oligomer-type phytosterol esters, specifically, a cosmetic composition for lips with excellent applicability due to its glossy and soft texture, such as a lip gloss composition although it is in the form of a solid stick and the inventive lip stick composition showed the superior glossy effect and soft use of solid sticks compared to conventional solid lipstick contents as well as the superior convenient effect on using stick products compared to liquid or semi-solid lip gloss contents when applied to lips, which is confirmed by several tests for example, on the sensual evaluation test of gloss and long-term stability by temperature, such as (1) Comparative measurement of sensual evaluation items (Experimental Example 1), (2) Comparative test on stability test (Experimental Example 2), and (3) Determination on compatibility of each ingredient (Experimental Example 3) etc.

In addition, inventive cosmetic composition can provide superior convenience in use by way of constructing an appropriate mixing composition ratio of lip gloss products in a solid stick form for the purpose of using a liquid or semi-solid form by mixing no wax at all or a small amount of wax in use.

Through the present invention, the present inventors have developed a new concept of ‘lip fondue’ formulation in a solid stick form having high gloss and a sense of use such as liquid or semi-solid lip gloss, which is called as “lip fondue formulation”, hereinafter, since it is a similar formulation such as a liquid melt of solid cheese.

According to one aspect, the present invention provides novel lip gloss-type cosmetic composition comprising (a) hydrocarbon-based wax purified from natural gas or petroleum with a melting point of 85° C. or higher, (b) oligomer-type phytosterol ester, (c) fatty acid complex ester oil, (d) polymeric film forming agent, and (e) a gelling agent including an organic modified clay mineral as essential components.

In a preferred embodiment, the present invention provides novel lip gloss-type cosmetic composition comprising (a) 1-9 weight part (w/w), preferably, 3-8 weight part (w/w), more preferably, 4-7 weight part (w/w) hydrocarbon-based wax purified from natural gas or petroleum with a melting point of 85° C. or higher, (b) 1-80 weight part (w/w), preferably, 10-50 weight part (w/w), more preferably, 20-40 weight part (w/w) oligomer-type phytosterol ester, (c) 1-80 weight part (w/w), preferably, 10-70 weight part (w/w), more preferably, 40-60 weight part (w/w) fatty acid complex ester oil, (d) 1-50 weight part (w/w), preferably, 5-30 weight part (w/w), more preferably, 7-20 weight part (w/w) polymeric film forming agent, and (e) 0.1-30 80 weight part (w/w), preferably, 0.5-20 weight part (w/w), more preferably, 1-10 weight part (w/w) a gelling agent including an organic modified clay.

The inventive composition may additionally comprise, not intended to limit thereto, “the other optional components” selected from the group consisting of at least one various pigment such as organic pigments, or inorganic pigments, a extender pigments, a moisturizer, a dispersant, an antioxidant, a sunscreen, perfume and/or the optional components depending on the purposed use.

The term “(a) hydrocarbon-based wax purified from natural gas or petroleum with a melting point of 85° C. or higher” disclosed herein comprises, for example, not intended to limit thereto, a polyethylene, ozokerite, ceresin, paraffin, microcrystalline wax, synthetic wax or one or more mixtures thereof, preferably, ozokerite, ceresin, synthetic wax or at least one mixture thereof, and most preferably, ‘Ceresin 9205’® of Koster Keunen Co. Ltd, with a melting point of 85-105° C. or ‘Lipwax PZ80-20’® of Japan Natural Co. Ltd, which can provides with several functions such as hardness to stick-type cosmetics, forming an outer structure etc.

The term “wax” which may be introduced into the cosmetic composition according to the present invention refers to a fatty substance having a reversible liquid/solid state change with an anisotropic crystal structure in a solid state and a liquid having a melting temperature of 80° C. or higher and generally less than 110° C., which can act on the structuring of a stick shape having minimal hardness and at least selected from polar wax, non-polar wax, or a mixture thereof.

In the present invention to solve the previous problems in the art due to the use of high content wax, for example, when the wax amount is 7.0% or more, the refractive index and transparency of the oil are affected to the extent that the gloss is not excellent, it is preferable that the content of using wax content in the inventive cosmetic composition, should be adjusted to 7.0% (w/w) or less, more preferably 6.0 to 7.0% (w/w) of the total composition content (100%, w/w), to provide an inventive product with excellent gloss and good usability on a solid stick by improving refractive index and transparency of oil

The term “(b) oligomer-type phytosterol ester” disclosed herein comprises, for example, not intended to limit thereto, a Bis-Behenyl/Isostearyl/Phytosteryl Dimer Dilinoleyl Dimer Dilinoleate, Bis-diglycerl polyacyladipate-2, Phytosteryl/Octyldodecyl Lauroyl Glutamate, Phytosteryl/Isostearyl/Cetyl/Stearyl/Behenyl Dimer Dilinoleate, Phytosteryl/Isostearyl/Cetyl/Stearyl/Behenyl Dimer Dilinoleate or at least one mixture thereof, preferably, a Bis-Behenyl/Isostearyl/Phytosteryl Dimer Dilinoleyl Dimer Dilinoleate, Bis-diglycerl polyacyladipate-2, or at least one mixture thereof, the most preferably, ‘Plandool-G’® of Nippon Fine Chemical Co. Ltd. (Bis-Behenyl/Isostearyl/Phytosteryl Dimer Dilinoleyl Dimer Dilinoleate), ‘Plandool-S or H’® of Nippon Fine Chemical Co. Ltd. (dimer ester of phytosteryl/Isostearyl/Cetyl/Stearyl/Behenyl Dimer Dilinoleate), which acts on the function of stick-type cosmetics with excellent applicability such as high gloss, good dispersibility of pigment, and good spreadability according to external temperature.

The term “(c) fatty acid complex ester oil” disclosed herein comprises, for example, not intended to limit thereto, a Pentaerythrityl Adipate/Caprate/Caprylate/Heptanoate, diisostearyl maleate, hydrogenated polyisobuden, Dipentaerythrityl hexahydroxystearate/hexastearate/hexarosinate, Dipentaerythrityl Hexahydroxystearate, Dipentaerythrityl Pentaisostearate, Dipentaerythrityl Hexahydroy Stearate/Tetra Stearate/Tetra Isostearate, Glyceryl Ethyl Hexanoate/Stearate/Adipate, Caprylic/Capric/Myristic/Stearic Triglyceride) or at least one mixture thereof, preferably, Pentaerythrityl Adipate/Caprate/Caprylate/Heptanoate, diisostearyl maleate, hydrogenated polyisobuden, or at least one mixture thereof, preferably, a Bis-Behenyl/Isostearyl/Phytosteryl Dimer Dilinoleyl Dimer Dilinoleate, Bis-diglycerl polyacyladipate-2, or at least one mixture thereof, which acts on the function of imparting an emolient effect and excellent gloss to the lips.

The term “(d) polymeric film forming agent” disclosed herein comprises, for example, not intended to limit thereto, a VP/Hexadecene Copolymer, Trimethylpentanediol/Adipic Acid/Glycerin Crosspolymer, Hydrogenated Polyisobutene & Ethylene/Propylene/Styrene Copolymer & Butylene/Ethylene/Styrene Copolymer, VP/Eicosene Copolymer, Bis-Dioctadecylamide Dimer Dilinoleic Acid/Ethylenediamine Copolymer, Cetearyl Dimethicone Crosspolymer, HDI/Trimethylol Hexyllactone Crosspolymer, Dimethicone/Vinyl Dimethicone Crosspolymer, Vinyl Dimethicone/Methicone Silsesquioxane Crosspolymer or at least one mixture thereof, preferably, VP/Hexadecene Copolymer, Trimethylpentanediol/Adipic Acid/Glycerin Crosspolymer, Hydrogenated Polyisobutene & Ethylene/Propylene/Styrene Copolymer & Butylene/Ethylene/Styrene Copolymer or at least one mixture thereof, which acts on the function of imparting a film formation, adhesion, and makeup persistence.

The term “(e) a gelling agent including an organic modified clay mineral ” disclosed herein comprises, for example, not intended to limit thereto, a Benton gel, Hectorite, Disteardimonium Hectorite, Quaternium-18 Hectorite, Stearalkonium Hectorite, Bentonite, Disteardimonium Bentonite, Quaternium-18 Bentonite, Dextrin Palmitate, Silica, Starch, Clay or at least one mixture thereof, preferably, Benton gel, more preferably, Benton gel prepared by the step of weighing the mixture of Disteardimonium Hectorite & Octyldodecanol & Propylene carbonate at the ratio of 10:87:3, and stirring them three times using by three-stage roll mill device before manufacturing the contents, of which process can also additionally mix Dextrin palmitate with the organic modified clay mineral after a pretreatment process of pre-heating, stirring, and mixing with a bentone gel in advance, which acts on the functions of increasing the viscosity of oil, improving the dispersibility of pigments, thixotropy of content properties, and improving thermal stability over time.

The inventive composition may additionally comprise, not intended to limit thereto, “one or more additional components selected from” selected from the group consisting of (f) dispersants, (g) moisturizers, (h) organic pigments, inorganic pigments, white pigments, extender pigments, or the mixture thereof, (i) fragrances, (j) antioxidants, or (k) ultraviolet blockers as the optionally addable components other than the above essential components depending on the purposed use within the range that does not impair the technical effect of the present invention.

In a preferred embodiment of the present invention, the present invention also provides novel lip gloss-type cosmetic composition comprising (a) hydrocarbon-based wax purified from natural gas or petroleum with a melting point of 85° C. or higher, (b) oligomer-type phytosterol ester, (c) fatty acid complex ester oil, (d) polymeric film forming agent, and (e) a gelling agent including an organic modified clay mineral as essential components; and at least one additional components” selected from the group consisting of (f) dispersants, (g) moisturizers, (h) organic pigments, inorganic pigments, white pigments, extender pigments, or the mixture thereof, (i) fragrances, (j) antioxidants, and (k) ultraviolet blockers as the optionally addable components.

The term “(f) dispersants” disclosed herein comprises, for example, not intended to limit thereto, Sorbitan Sesquioleate, Sorbitan Isostearate, Sorbitan Stearate, Sorbitan Oleate, Cetyl PEG/PPG-10/1 Dimethicone, Bis-PEG/PPG-14/14 Dimethicone/Dimethicone, PEG-10 Dimethicone or at least one mixture thereof

The term “(g) moisturizers” disclosed herein comprises, for example, not intended to limit thereto, Tocopheryl Acetate, Ceramide, Phyto Sphingosine, Bisaborol or at least one mixture thereof

The term “(h) organic pigments, inorganic pigments, white pigments, extender pigments, or the mixture thereof” disclosed herein comprises, for example, not intended to limit thereto, organic pigments such as Red No. 104 (1) Red No. 202, Red No. 218, Red No. 227, Red No. 40, Yellow No. 4, Yellow No. 5, Blue No. 1 and the like; inorganic pigments such as red iron oxide, yellow iron oxide, black iron oxide, ultramarine and the like; white pigments such as titan dioxide, zinc oxide and the like ; extender pigments such as Mica, Sericite, Talc, Silica and the like or at least one mixture thereof.

The term “(i) fragrances” disclosed herein comprises, for example, not intended to limit thereto, synthetic fragrances, natural fragrances, combination fragrances, Essential oil, Peppermint Oil, Menthoxypropanediol or at least one mixture thereof.

The term “(j) antioxidants” disclosed herein comprises, for example, not intended to limit thereto, 1,2-hexandiol, phenoxyethanol, BHT(2,6-Di-tert-butyl-4-methylphenol), dehydroacetic acid, ethylhexyl glycerin or at least one mixture thereof.

The term “(k) ultraviolet blockers” disclosed herein comprises, for example, not intended to limit thereto, ethylhexyl methoxycinnamate, 4-Methylbenzylidene Camphor, Isoamyl p-Methoxycinnamate, Butyl Methoxydibenzoylmethane, ethylhexyl salicylate, Ethylhexyl Dimethyl PABA, Benzophenone-3, Benzophenone-4, Benzophenone-8 or at least one mixture thereof.

In a preferred embodiment of the present invention, the present invention also provides novel lip gloss-type cosmetic composition comprising (a) 1-9 weight part (w/w), preferably, 3-8 weight part (w/w), more preferably, 4-7 weight part (w/w) hydrocarbon-based wax purified from natural gas or petroleum with a melting point of 85° C. or higher, (b) 1-80 weight part (w/w), preferably, 10-50 weight part (w/w), more preferably, 20-40 weight part (w/w) oligomer-type phytosterol ester, (c) 1-80 weight part (w/w), preferably, 10-70 weight part (w/w), more preferably, 40-60 weight part (w/w) fatty acid complex ester oil, (d) 1-50 weight part (w/w), preferably, 5-30 weight part (w/w), more preferably, 7-20 weight part (w/w) polymeric film forming agent, and (e) 0.1-30 80 weight part (w/w), preferably, 0.5-20 weight part (w/w), more preferably, 1-10 weight part (w/w) a gelling agent including an organic modified clay mineral as essential components; and at least one additional components” selected from the group consisting of (00.01-10 weight part (w/w), preferably, 0.03-5 weight part (w/w), more preferably, 0.1-2 weight part (w/w) dispersants, (g) 0.01-10 weight part (w/w), preferably, 0.03-5 weight part (w/w), more preferably, 0.05-2 weight part (w/w) moisturizers, (h) 0.1-30 weight part (w/w), preferably, 0.5-20 weight part (w/w), more preferably 1-10 weight part (w/w) organic pigments, inorganic pigments, white pigments, extender pigments, or the mixture thereof, (i) 0.01-5 weight part (w/w), preferably, 0.03-3 weight part (w/w), more preferably, 0.05-1 weight part (w/w) fragrances, (j) 0.01-5 weight part (w/w), preferably, 0.03-3 weight part (w/w), more preferably, 0.05-2 weight part (w/w) antioxidants, and (k) 0.01-20 weight part (w/w), preferably, 0.03-10 weight part (w/w), more preferably, 0.05-8 weight part (w/w) ultraviolet blockers as the optionally addable components.

The composition of the present invention may be prepared through a conventional pre-treatment process such as heating, mixing, stirring, degassing, charging, and cooling according to the conventional preparation method well known in the art.

For example, the inventive composition of the present invention can be prepared by follows;

The composition of the present invention shall be performed on condition that all the component shall be accurately weighed so that there is no loss due to weighting and the melting point of the composition is maintained at 60° C. or higher during the entire process, and;

The composition of the present invention may be prepared by the process comprising steps of; (1) weighing each components such as (a) 1-9 weight part (w/w), preferably, 3-8 weight part (w/w), more preferably, 4-7 weight part (w/w) hydrocarbon-based wax purified from natural gas or petroleum with a melting point of 85° C. or higher, (b) 1-80 weight part (w/w), preferably, 10-50 weight part (w/w), more preferably, 20-40 weight part (w/w) oligomer-type phytosterol ester, (c) 1-80 weight part (w/w), preferably, weight part (w/w), more preferably, 40-60 weight part (w/w) fatty acid complex ester oil, (d) 1-50 weight part (w/w), preferably, 5-30 weight part (w/w), more preferably, 7-20 weight part (w/w) polymeric film forming agent, and (e) 0.1-30 80 weight part (w/w), preferably, 0.5-20 weight part (w/w), more preferably, 1-10 weight part (w/w) a gelling agent including an organic modified clay mineral, and stirring at the speed of 500 to 3,000 RPM, more preferably, 1,000 to 2,000 RPM and at the temperature ranging from 60 to 100° C., more preferably, 80 to 95° C., to dissolve all the mixture thoroughly and obtain the 1^{st} mixture at 1^{st} step; (2) subjecting next process including a coloring and degassing step in the stirred 1^{st} mixture at the first step to obtain 2^{nd} mixture at 2^{nd} step; (3) introducing the 2^{nd} mixture prepared in step 2 into a heating charger, heating at the temperature range of 70 to 100° C., preferably 80 to 95° C., dissolving the mixture and directly charging the dissolved mixture in the product container to afford the 1^{st} charged stick filler at 3^{rd} step; (4) melting the surface of the charged stick at 3^{rd} step with a post-heat in the range of 200 to 500° C., preferably 300 to 400° C. and performing the 2^{nd} charging step to afford the 2^{nd} charged stick filler at 4^{th} step; (5) naturally cooling the 2^{nd} charged stick filler at room temperature for 2 to 20 minutes, preferably 3 to 5 minutes, dissolving the cooled stick surface again with a post-heat unit of 200 to 500° C., preferably 300 to 400° C. and cooling the heated stick filler at room temperature for 2 to 20 minutes, preferably 5 to 10 minutes at 5^{th} step to afford the final inventive lip gloss stick cosmetic composition, which is named as “lip fondue formulation”, hereinafter.

It is another object of the present invention to provide a process for preparing novel lip gloss-type cosmetic composition and the novel lip gloss-type cosmetic composition prepared thereby.

The alternative process for preparing inventive composition and/or the alternative composition can be obvious in light of the technical level of a person with ordinary knowledge in the art and if necessary, can be obtained by conventional methods and composition disclosed in the literature ( [symbol] (new cosmetology), / [symbol] (Mitusi takeo), dongwha Technique Press, 1^{st} Ed., 2004).

It is another object of the present invention to provide novel lip gloss-type cosmetic composition, preferably, wherein the molding stick has physical properties of which melting point in the range of 50 to 80° C., desirably 60 to 70° C., and hardness in the range of 20 to 100 g/f, desirably 30 to 90 g/f, more desirably 40 to 80 g/f (Fudoh Rheometer, Pin No. 30, settling speed of 2 sec/min, Range 200/2N, depth 21 mm).

It is another object of the present invention to provide novel lip gloss-type cosmetic composition in the form of a glossy stick including a lip gloss, lip balm, lipstick, lip treatment, lip care products and the like.

In this invention, instead of a wax raw material that determines the hardness of a general lipstick, since the high content (more than 40% w/w) of semi-solid (paste) type oligomer type phytosterol ester (for example, Plandool-G) which changes to a liquid phase at more than 40° C., forms a structure of the lipstick;
(1) the combination of oil components with excellent compatibility except for components having hydroxy-group or glyceryl-group and hydrogenated among the emoliant components, is the most essential since the stabilization of the oligomer type phytosterol ester is affected by the long-term stability due to temperature,
(2) In consideration of the characteristics of high content oligomer-type phytosterol (Plandool-G), which melts at 40° C. and changes its properties, the overall process shall thoroughly be managed so that the melting point of the bulk is 60° C. or higher even in manufacture.
(3) Even in the stick molding process, the conditions of the cooling and heating

processes are managed so that the crystals of the wax are formed as the outer layer of the stick.

It will be apparent to those skilled in the art that various modifications and variations can be made in the compositions, use and preparations of the present invention without departing from the spirit or scope of the invention

The present invention is more specifically explained by the following examples. However, it should be understood that the present invention is not limited to these examples in any manner.

### Advantageous Effects of Invention

As described above, the composition of the present invention exhibits superior oil gloss and soft solid stick usage compared to conventional solid lipstick contents when applied to lips, and convenience of using stick products compared to liquid or semi-solid lip gloss contents, which is confirmed by several tests for example, on the sensual evaluation test of gloss and long-term stability by temperature, such as (1) Comparative measurement of sensual evaluation items (Experimental Example 1), (2) Comparative test on stability test (Experimental Example 2), and (3) Determination on compatibility of each ingredient (Experimental Example 3); specifically, the inventive composition provides a cosmetic composition for lips with excellent applicability due to its glossy and soft texture, such as a lip gloss composition although it is in the form of a solid stick, and it is possible to construct an appropriate mixing composition ratio of lip gloss products for liquid or semi-solid use by mixing no wax or a small amount of wax, and to structure them in a solid stick form to give convenience in lip makeup.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Examples and Experimental Examples are intended to further illustrate the present invention without limiting its scope.

### Comparative Example 1. The Preparation of Comparative Lipstick Composition (1-3)

The comparative lip gloss and lipstick composition (See Table 1) which has previously known to show high gloss, were prepared to compare their sense of use and gloss with the inventive composition (See Table 2).

The comparative lip gloss and lipstick composition

TABLE 1

| | | Comparative Examples* | | |
|---|---|---|---|---|
| category | Ingredient | CE 1 | CE 2 | CE 3 |
| (a)Solid wax | Polyethylene | 6.00 | 2.70 | 5.25 |
| | Micro-crystalline wax | — | 3.50 | 2.50 |
| | Paraffin/Micro-crystalline wax | — | 4.00 | — |
| | Synthetic wax/ethylene/propylene copolymer | — | 2.50 | — |
| | Sheer butter | 3.00 | — | — |
| (b)paste oil | Bis-Behenyl/isosteary/phytosteryl dimer | — | 3.00 | — |
| | di-linoleyl dimer di-linolate | | | |
| | Bis-diglyceryl polyacyladipate-2 | 7.60 | — | 7.49 |
| | Petroleum | 15.00 | — | — |
| | stearoxymethicone/Dimethicone crosspolymer | — | — | 5.00 |
| (c) liquid oil | Pentaerythrutyl adipate/caprate/caprylate/hepanoate | — | — | 32.732 |
| | Diisostearyl maleate | 17.50 | — | — |
| | Myristyl stearate/myristyl palmitate | 4.50 | — | — |
| | Pentaerythrityl tetraethyl hexanoate | — | 6.30 | — |
| | Neopentyl glycol dicaprate | — | 10.00 | — |
| | caprylic/capric triglyceride | — | 10.00 | — |
| | Propylene glycol dibenzoate | — | 12.00 | — |
| | isononyl isononanoate | — | 6.00 | — |
| | Octyl dodecanol | 16.00 | — | 19.10 |
| | Polybuden | 25.52 | — | 2.50 |
| | hydrogenated polybuden | — | 4.00 | — |
| | Hydrogenated di-isolinoleyl alcohol | — | — | 5.90 |
| | Diphenyl dimethicone | — | 5.00 | — |
| | Dimethicone | — | 10.00 | — |
| (d) Film- | Phenylpropyl dimethyl siloxysilicate | — | 2.00 | — |
| Forming | Hydrogenated styrene/methyl styrene/indene copolymer | — | 2.70 | — |
| Agents | | | | |
| (d)gelling | Silica silicate | 0.80 | — | — |
| agent | Ethyl cellulose | — | — | 1.56 |
| | Silica | — | — | 3.50 |
| | Methyl glucose sesquistearate | — | — | 0.79 |
| (e) humectants | surfactant | — | 2.00 | 0.664 |
| (f)antiseptic | preservative | 0.05 | 0.10 | 0.15 |
| (e)moisturizer | Additive | — | — | 0.7 |
| (f) dye | Pigment red 202 | 4.00 | 14.20 | 12.164 |
| (g)perfume | Combination fragrance | 0.03 | — | — |

| | | | |
|---|---|---|---|
| sum | 100.00 | 100.00 | 100.00 |

*CE 1: Comparative Example 1/CE 2: Comparative Example 2/CE 3: Comparative Example 3

### Example 1. The Preparation of Inventive Lipstick (Lip-Fondue) Cosmetic Composition (1)-(4)

When weighing in this experiment, the melting point of the composition must be 60° C. or higher, so that there is no loss due to weight loss (LOSS). The wax and gelling agents in the experiment must be weighed separately to afford an accurate blending. Each raw material listed in Table 2 (the amount was based on weight percent (%)) was weighed in a beaker, heated and dissolved at 80-95° C. in a molten bath (SUS vacuum tank, Packingman machine Co. Ltd, Hwasung city, Korea) to which a dispenser was attached, stirred at 1,000-2,000 RPM, and then the final mixture was prepared through a coloring and degassing process. The produced final mixture was introduced into a heating charger (lipstick automatic molding machine, Woojung Tech, Korea), heated and dissolved at 80-95° C. to directly perform primary charging to the final product container. The surface of the charged stick was melted with a heating charger at 300-400° C. as a post-heat, and then the stick was charged again and cooled naturally for 3-5 min at room temperature. After melting the cooled stick surface again with a post-heat of 300-400° C., the final product was prepared through a secondary cooling process at room temperature for 5-10 minutes (designated as “inventive lip-fondue lipstick” hereinafter).

TABLE 2
inventive lipstick (lip fondue) composition

| | | Example (weight %)* | | | |
|---|---|---|---|---|---|
| category | ingredients | EX 1 | EX 2 | EX 3 | EX 4 |
| (a) Hydrocarbon- | Ceresin | 6.00 | 6.00 | 6.00 | 6.00 |
| based wax | Synthetic wax/ethylene/propylene copolymer | 0.50 | 0.50 | 0.50 | 0.50 |
| obtained by the | | | | | |
| purification of | | | | | |
| a natural gas | | | | | |
| or petroleum | | | | | |
| having a melting | | | | | |
| point of 85° | | | | | |
| C. or higher | | | | | |
| (b) Oligomeric | Bis-Behenyl/isoateary/phytosteryl | 25.00 | 10.00 | 10.00 | 10.00 |
| phytosterol | dimer dilinoleyl dimer dilinolate | | | | |
| ester | Bis-diglyceryl polyacyladipate-2 | — | 15.00 | 15.00 | 15.00 |
| (c) Synthetic | Diisostearyl maleate | 37.77 | 32.77 | 32.77 | 32.77 |
| ester oil with | Pentaerythrityl adipate/caprate/caprylate/heptanoate | 15.00 | 15.00 | 15.00 | 15.00 |
| fatty acids | hydrogenated polyisobuden | — | 5.00 | 5.00 | 5.00 |
| (d) Film-forming | VP/Hexadecene Copolymer | 10.00 | — | 10.00 | — |
| agent of high | Trimethylpentanediol/Adipic Acid/Glycerin Crosspolymer | — | 10.00 | — | — |
| polymer | Hydrogenated Polyisobutene & Ethylene/Propylene/Styrene | — | — | — | 10.00 |
| | Copolymer & Butylene/Ethylene/Styrene Copolymer | | | | |
| (e) gelling | Disteardimonium Hectorite/octyldodecanol/propylene | 3.00 | 3.00 | 3.00 | 3.00 |
| agent including | carbonate (mixed ratio = 10:87:3) | | | | |
| organic modified | dextrin palmitate | 0.03 | 0.03 | 0.03 | 0.03 |
| clay minerals | | | | | |
| (f)dispersants | Sorbitan Sesquioleate | 0.50 | 0.50 | 0.50 | 0.50 |
| (g)emollients | tocopheryl acetate | 0.10 | 0.10 | 0.10 | 0.10 |
| (h)pigment | Pigment red 202 | 1.00 | 1.00 | 1.00 | 1.00 |
| | Pigment yellow 5 | 1.00 | 1.00 | 1.00 | 1.00 |
| (i)perfume | Combination perfume | 0.10 | 0.10 | 0.10 | 0.10 |

| | | | | |
|---|---|---|---|---|
| sum | 100.00 | 100.00 | 100.00 | 100.00 |

*EX 1: Example 1/EX 2: Example 2/EX 3: Example 3/EX 4: Example 4

### Experimental Example 1. Comparative Measurement of Sensual Evaluation Items

In order to evaluate the sensual items on the inventive lipstick composition and comparative lipstick prepared in Examples and Comparative Examples, following product quality evaluation test on the sense of use and gloss was performed.

Each test samples were spread on the lip of volunteers consisting of 30 women, more than three times a day and the preference with respect to two items, i.e., (a) the sense of use and (b) the sense of gloss, was evaluated.

The test result shown in Table 3 was scored (maximum score: 10) and classified into five scores, i.e., very good (10), good (8), common (6), bad (4) and very bad (2) to be total score of 10.

TABLE 3
product quality evaluation test

| | | | | | Comparative | | |
|---|---|---|---|---|---|---|---|
| | Examples* | | | | Examples** | | |
| Evaluation item | EX 1 | EX 2 | EX 3 | EX 4 | CE 1 | CE2 | CE3 |
| (a) the sense of use | 10 | 6 | 6 | 6 | 4 | 4 | 4 |
| (b) the sense of gloss | 8 | 8 | 8 | 4 | 6 | 4 | 6 |
| Mean | 18 | 14 | 14 | 10 | 10 | 8 | 10 |

*EX 1: Example 1/EX 2: Example 2/EX 3: Example 3/EX 4: Example 4
**CE 1: Comparative Example 1/CE 2: Comparative Example 2/CE 3: Comparative Example 3

As can be seen in Table 3, the inventive lip gloss stick type compositions showed unpredictably higher scores on the (a) the sense of use and (b) the sense of gloss comparing with comparative lipstick composition.

Accordingly, it has been confirmed that the various kind of inventive lipstick composition prepared in Examples unpredictably higher scores on the (a) the sense of use and (b) the sense of gloss comparing with comparative lipstick composition known in the art.

### Experimental Example 2. Comparative Test on Stability

In order to determine the stability of the inventive lipstick composition and comparative lipstick prepared in Examples and Comparative Examples, following stability test was performed.

The basic physical property such as stability according to the various storage temperature of inventive lipstick composition and comparative lipstick composition prepared in Examples and Comparative Examples, was determined for two months, varying with the storage temperature of 4° C., 25° C., 40° C., 45° C., 50° C., −20° C., Cycle (−10° C.-45° C.) to compare the stability with respect to the hardness of each product between the inventive lipstick composition and comparative lipstick composition.

As can be seen in Tables 4-9, the inventive lip gloss stick type compositions showed very little change with the passage of time and stable appearance condition according to the various temperature.

TABLE 4
change with the passage of time and the hardness of
the composition (at 25° C. of storage temperature)

| 25° C. | 0 week | 1 week | 2 weeks | 4 weeks | 8 weeks |
|---|---|---|---|---|---|
| Example 1 | 48 | 47 | 49 | 43 | 46 |
| Example 2 | 37 | 31 | 30 | 45 | 26 |
| Example 3 | 37 | 37 | 27 | 35 | 31 |
| Example 4 | 48 | 27 | 31 | 26 | 8 |

TABLE 5
change with the passage of time and the hardness of
the composition (at 40° C. of storage temperature)

| 40° C. | 0 week | 1 week | 2 weeks | 4 weeks | 8 weeks |
|---|---|---|---|---|---|
| Example 1 | 48 | 35 | 52 | 52 | 43 |
| Example 2 | 37 | 33 | 29 | 59 | 33 |
| Example 3 | 37 | 34 | 22 | 38 | 27 |
| Example 4 | 48 | 40 | 48 | 43 | 26 |

TABLE 6
change with the passage of time and the hardness of
the composition (at 50° C. of storage temperature)

| 50° C. | 0 week | 1 week | 2 weeks | 4 weeks | 8 weeks |
|---|---|---|---|---|---|
| Example 1 | 48 | 61 | 46 | 59 | 48 |
| Example 2 | 37 | 41 | 34 | 74 | 42 |
| Example 3 | 37 | 35 | 26 | 36 | 31 |
| Example 4 | 48 | 45 | 67 | 51 | 32 |

TABLE 7
change with the passage of time and the hardness of
the composition (at 4° C. of storage temperature)

| 4° C. | 0 week | 1 week | 2 weeks | 4 weeks | 8 weeks |
|---|---|---|---|---|---|
| Example 1 | 48 | 48 | 49 | 39 | 41 |
| Example 2 | 37 | 31 | 14 | 25 | 15 |
| Example 3 | 37 | 34 | 16 | 25 | 18 |
| Example 4 | 48 | 24 | 33 | 27 | 6 |

TABLE 8
change with the passage of time and the hardness of the composition
(at −20° C. of storage temperature)

| −20° C. | 0 week | 1 week | 2 weeks | 4 weeks | 8 weeks |
|---|---|---|---|---|---|
| Example 1 | 48 | 49 | 51 | 41 | 37 |
| Example 2 | 37 | 33 | 20 | 38 | 25 |
| Example 3 | 37 | 34 | 22 | 33 | 28 |
| Example 4 | 48 | 24 | 33 | 23 | 5 |

TABLE 9
change with the passage of time and the hardness of the composition
(at −10° C.~45° C. of storage temperature)

| Cycle | | | | | |
|---|---|---|---|---|---|
| (−10° C.~45° C.) | 0 week | 1 week | 2 weeks | 4 weeks | 8 weeks |
| Example 1 | 48 | 43 | 46 | 49 | 39 |
| Example 2 | 37 | 31 | 16 | 44 | 28 |
| Example 3 | 37 | 34 | 18 | 34 | 31 |
| Example 4 | 48 | 41 | 40 | 38 | 22 |

As can be shown in the above Tables, it has been confirmed that the cosmetic composition of the lip gloss stick prepared according to the present invention, maintained a constant hardness over time and stabilized appearance under all storage temperature conditions

### Experimental Example 3. Determination on Compatibility of Each Ingredient

To determine the compatibility of each ingredient in inventive products, following comparison test on observing the appearance condition of final produce prepared by mixing each wax and oil with various mixed ratio and heating with stirring to prepare final products, has been performed according to the method well known in the art and the test result was shown in Table 10.

TABLE 10
Determination on the compatibility of each ingredient

| | | Test |
|---|---|---|
| NO | Ingredients | result |
| 1 | ceresin:Diisostearyl maleate:Bis-Behenyl/isoateary/phytosteryl | ◯ |
| | dimer dilinoleyl dimer dilinolate (10:60:30) | |
| 2 | ceresin:Diisostearyl maleate:Bis-diglyceryl polyacyladipate-2 (10:60:30) | X |
| 3 | ceresin:Diisostearyl maleate: (20:80) | ◯ |
| 4 | ceresin; Pentaerythrityl adipate/caprate/caprylate/heptanoate: (20:80) | ◯ |
| 5 | ceresin; Hydrogenated Polyisobutene (20:80) | X |
| 6 | ceresin; VP/Hexadecene Copolymer (20:80) | ◯ |
| 7 | ceresin; Trimethylpentanediol/Adipic Acid/Glycerin Crosspolymer (20:80) | X |
| 8 | ceresin; Hydrogenated Polyisobutene:Ethylene/Propylene/Styrene | X |
| | Copolymer & Butylene/Ethylene/Styrene Copolymer (10:60:30) | |

*◯: good compatibility, X: bad compatibility (separation phenomenon occurred)

TABLE 11
Comprehensive evaluation results.

| TESTING | Examples* | | | Comparative Examples** | | |
|---|---|---|---|---|---|---|
| CONTENT | EX1 | EX2 | EX3 | CE1 | CE2 | CE3 |
| Sense of use | 8 | 10 | 8 | 6 | 6 | 6 |
| Sense of gloss | 6 | 8 | 6 | 8 | 8 | 4 |
| Stability | 7 | 8 | 8 | 4 | 4 | 4 |
| Mean | 7.0 | 8.7 | 7.3 | 6.0 | 6.0 | 4.7 |

*EX 1: Example 1/EX 2: Example 2/EX 3: Example 3/EX 4: Example 4
**CE 1: Comparative Example 1/CE 2: Comparative Example 2/CE 3: Comparative Example 3

As shown in Table 11, it has been confirmed that the lip gloss stick cosmetic compositions prepared according to the present invention are found to show the excellent test result in terms of sense of use, gloss, and stability.

As a result of the experiment, it was confirmed that when components having hydrophilic properties with glyceryl structural formula or hydrogenated structural formula were mixed, the hardness of the stick was degraded or the expansion or contraction of the stick was affected through the above high-temperature stability evaluation results, cause by their hygroscopic properties

### MODE FOR THE INVENTION

Hereinafter, the formulating methods and kinds of components will be described, but the present invention is not limited to them. The representative preparation examples were described as follows.

The inventive novel lip gloss-type cosmetic composition may be prepared in the form of a glossy stick including a lip gloss, lip balm, lipstick, lip treatment, lip care products and the like.

The invention being thus described as will be obvious that it may be varied in many ways. Such variations are not to be regarded as a departure from the spirit and scope of the present invention, and all such modifications as would be obvious to those skilled in art are intended to be included within the scope of the following claims.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described above, the composition of the present invention exhibits superior oil gloss and soft solid stick usage compared to conventional solid lipstick contents when applied to lips, and convenience of using stick products compared to liquid or semi-solid lip gloss contents, which is confirmed by several tests for example, on the sensual evaluation test of gloss and long-term stability by temperature, such as (1) Comparative measurement of sensual evaluation items (Experimental Example 1), (2) Comparative test on stability test (Experimental Example 2), and (3) Determination on compatibility of each ingredient (Experimental Example 3); specifically, the inventive composition provides a cosmetic composition for lips with excellent applicability due to its glossy and soft texture, such as a lip gloss composition although it is in the form of a solid stick, and it is possible to construct an appropriate mixing composition ratio of lip gloss products for liquid or semi-solid use by mixing no wax or a small amount of wax, and to structure them in a solid stick form to give convenience in lip makeup.

1. A lip gloss-type cosmetic composition comprising (a) hydrocarbon-based wax purified from natural gas or petroleum with a melting point of 85° C. or higher, (b) oligomer-type phytosterol ester, (c) fatty acid complex ester oil, (d) polymeric film forming agent, and (e) a gelling agent including an organic modified clay mineral as essential components.

2. The lip gloss-type cosmetic composition according to claim 1, Wherein said lip gloss-type cosmetic composition comprising (a) 1-9 weight part (w/w) hydrocarbon-based wax purified from natural gas or petroleum with a melting point of 85° C. or higher, (b) 1-80 weight part (w/w) oligomer-type phytosterol ester, (c) 1-80 weight part (w/w) fatty acid complex ester oil, (d) 1-50 weight part (w/w) polymeric film forming agent, and (e) 0.1-30 weight part (w/w) a gelling agent including an organic modified clay.

3. The lip gloss-type cosmetic composition according to claim 1, Wherein said “(a) hydrocarbon-based wax purified from natural gas or petroleum with a melting point of 85° C. or higher” is selected from a polyethylene, ozokerite, ceresin, paraffin, microcrystalline wax, synthetic wax or one or more mixtures thereof.

4. The lip gloss-type cosmetic composition according to claim 1, Wherein said “(b) oligomer-type phytosterol ester” is selected from a Bis-Behenyl/Isostearyl/Phytosteryl Dimer Dilinoleyl Dimer Dilinoleate, Bis-diglycerl polyacyladipate-2, Phytosteryl/Octyldodecyl Lauroyl Glutamate, Phytosteryl/Isostearyl/Cetyl/Stearyl/Behenyl Dimer Dilinoleate, Phytosteryl/Isostearyl/Cetyl/Stearyl/Behenyl Dimer Dilinoleate or at least one mixture thereof.

5. The lip gloss-type cosmetic composition according to claim 1, Wherein said “(c) fatty acid complex ester oil” is selected from a Pentaerythrityl Adipate/Caprate/Caprylate/Heptanoate, diisostearyl maleate, hydrogenated polyisobuden, Dipentaerythrityl hexahydroxystearate/hexastearate/hexarosinate, Dipentaerythrityl Hexahydroxystearate, Dipentaerythrityl Pentaisostearate, Dipentaerythrityl Hexahydroy Stearate/Tetra Stearate/Tetra Isostearate, Glyceryl Ethyl Hexanoate/Stearate/Adipate, Caprylic/Capric/Myristic/Stearic Triglyceride) or at least one mixture thereof.

6. The lip gloss-type cosmetic composition according to claim 1, Wherein said “(d) polymeric film forming agent” is selected from a VP/Hexadecene Copolymer, Trimethylpentanediol/Adipic Acid/Glycerin Crosspolymer, Hydrogenated Polyisobutene & Ethylene/Propylene/Styrene Copolymer & Butylene/Ethylene/Styrene Copolymer, VP/Eicosene Copolymer, Bis-Dioctadecylamide Dimer Dilinoleic Acid/Ethylenediamine Copolymer, Cetearyl Dimethicone Crosspolymer, HDI/Trimethylol Hexyllactone Crosspolymer, Dimethicone/Vinyl Dimethicone Crosspolymer, Vinyl Dimethicone/Methicone Silsesquioxane Crosspolymer or at least one mixture thereof

7. The lip gloss-type cosmetic composition according to claim 1, Wherein said “ (e) a gelling agent including an organic modified clay mineral ” is selected from a Benton gel, Hectorite, Disteardimonium Hectorite, Quaternium-18 Hectorite, Stearalkonium Hectorite, Bentonite, Disteardimonium Bentonite, Quaternium-18 Bentonite, Dextrin Palmitate, Silica, Starch, Clay or at least one mixture thereof.

8. A cosmetic composition comprising (a) hydrocarbon-based wax purified from natural gas or petroleum with a melting point of 85° C. or higher, (b) oligomer-type phytosterol ester, (c) fatty acid complex ester oil, (d) polymeric film forming agent, and (e) a gelling agent including an organic modified clay mineral as essential components; and at least one additional components” selected from the group consisting of (f) dispersants, (g) moisturizers, (h) organic pigments, inorganic pigments, white pigments, extender pigments, or the mixture thereof, (i) fragrances, (j) antioxidants, and (k) ultraviolet blockers as the optionally addable components.

9. The lip gloss-type cosmetic composition according to claim 8, Wherein said lip gloss-type cosmetic composition comprises (a) 1-9 weight part (w/w) hydrocarbon-based wax purified from natural gas or petroleum with a melting point of 85° C. or higher, (b) 1-80 weight part (w/w) oligomer-type phytosterol ester, (c) 1-80 weight part (w/w) fatty acid complex ester oil, (d) 1-50 weight part (w/w) polymeric film forming agent, and (e) 0.1-30 weight part (w/w) a gelling agent including an organic modified clay mineral as essential components; and at least one additional components selected from the group consisting of (f) 0.01-10 weight part (w/w) dispersants, (g) 0.01-10 weight part (w/w) moisturizers, (h) 0.1-30 weight part (w/w) organic pigments, inorganic pigments, white pigments, extender pigments, or the mixture thereof, (i) 0.01-5 weight part (w/w) fragrances, (j) 0.01-5 weight part (w/w) antioxidants, and (k) 0.01-20 weight part (w/w) ultraviolet blockers as the optionally addable components,

10. The lip gloss-type cosmetic composition according to claim 8, Wherein said “(f) dispersants” is selected from Sorbitan Sesquioleate, Sorbitan Isostearate, Sorbitan Stearate, Sorbitan Oleate, Cetyl PEG/PPG-10/1 Dimethicone, Bis-PEG/PPG-14/14 Dimethicone/Dimethicone, PEG-10 Dimethicone or at least one mixture thereof.

11. The lip gloss-type cosmetic composition according to claim 8, Wherein said “(g) moisturizers” is selected from Tocopheryl Acetate, Ceramide, Phyto Sphingosine, Bisaborol or at least one mixture thereof.

12. The lip gloss-type cosmetic composition according to claim 8, Wherein said “(h) organic pigments, inorganic pigments, white pigments, extender pigments, or the mixture thereof” is selected from organic pigments such as Red No. 104 (1), Red No. 202, Red No. 218, Red No. 227, Red No. 40, Yellow No. 4, Yellow No. 5, Blue No. 1 and the like; inorganic pigments such as red iron oxide, yellow iron oxide, black iron oxide, ultramarine and the like; white pigments such as titan dioxide, zinc oxide and the like;
extender pigments such as Mica, Sericite, Talc, Silica and the like or at least one mixture thereof.

13. The lip gloss-type cosmetic composition according to claim 8, Wherein said “(i) fragrances” is selected from synthetic fragrances, natural fragrances, combination fragrances, Essential oil, Peppermint Oil, Menthoxypropanediol or at least one mixture thereof.

14. The lip gloss-type cosmetic composition according to claim 8, Wherein said “(j) antioxidants” is selected from 1,2-hexandiol, phenoxyethanol, BHT(2,6-Di-tert-butyl-4-methylphenol), dehydroacetic acid, ethylhexyl glycerin or at least one mixture thereof.

15. The lip gloss-type cosmetic composition according to claim 8, Wherein said “(k) ultraviolet blockers” is selected from ethylhexyl methoxycinnamate, 4-Methylbenzylidene Camphor, Isoamyl p-Methoxycinnamate, Butyl Methoxydibenzoylmethane, ethylhexyl salicylate, Ethylhexyl Dimethyl PABA, Benzophenone-3, Benzophenone-4, Benzophenone-8 or at least one mixture thereof.

16. The lip gloss-type cosmetic composition according to claim 1, Wherein said molding stick has physical properties of which melting point in the range of 50 to 80° C. and hardness in the range of 20 to 100 g/f.

17. The lip gloss-type cosmetic composition according to claim 1, Wherein said cosmetic composition is in the form of a glossy stick including a lip gloss, lip balm, lipstick, lip treatment, lip care products and the like.

18. A method for preparing The lip gloss-type cosmetic composition according to claim 1 comprising steps of; (1) weighing (a) 1-9 weight part (w/w) hydrocarbon-based wax purified from natural gas or petroleum with a melting point of 85° C. or higher, (b) 1-80 weight part (w/w) oligomer-type phytosterol ester, (c) 1-80 weight part (w/w) fatty acid complex ester oil, (d) 1-50 weight part (w/w) polymeric film forming agent, and (e) 0.1-30 weight part (w/w) a gelling agent including an organic modified clay mineral, and stirring at the speed of 500 to 3,000 RPM and at the temperature ranging from 60 to 100° C. to dissolve all the mixture thoroughly and obtain the 1^{st} mixture at 1^{st} step; (2) subjecting next process including a coloring and degassing step in the stirred 1^{st} mixture at the first step to obtain 2^{nd} mixture at 2^{nd} step; (3) introducing the 2^{nd} mixture prepared in step 2 into a heating charger, heating at the temperature range of 70 to 100° C., dissolving the mixture and directly charging the dissolved mixture in the product container to afford the 1^{st} charged stick filler at 3^{rd} step; (4) melting the surface of the charged stick at 3^{rd} step with a post-heat in the range of 200 to 500° C. and performing the 2^{nd} charging step to afford the 2^{nd} charged stick filler at 4^{th} step; (5) naturally cooling the 2^{nd} charged stick filler at room temperature for 2 to 20 minutes dissolving the cooled stick surface again with a post-heat unit of 200 to 500° C. and cooling the heated stick filler at room temperature for 2 to 20 minutes at 5^{th} step to afford the final inventive lip gloss stick cosmetic composition.
